(19) United States
(12) Patent Application Publication
(10) Pub. No.: US 2007/0233166 A1
(43) Pub. Date: Oct. 4, 2007

(54) Low-Cost Lancing Device with Cantilevered Leaf Spring for Launch and Return

(75) Inventor: Jeffrey T. Stout, Smyrna, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Marietta, GA (US)

(21) Appl. No.: 11/569,495

(22) PCT Filed: May 26, 2005
(86) PCT No.: PCT/US05/18866

§ 371 Date: Nov. 21, 2006

Related U.S. Application Data
(60) Provisional application No. 60/574,779, filed on May 27, 2004.

(51) Int. Cl. A61B 17/14

(52) U.S. Cl. 606/182

(57) ABSTRACT

A lancing assembly is pivotally coupled to a housing and includes an actuator and a leaf spring carrying a lancet. When the actuator is in its set position, the leaf spring and lancet are in their set position with the leaf spring engaging a catch member of the housing. When the actuator is moved to its actuated position, the leaf spring pivots and deflects against the catch member, thereby storing a drive charge. The leaf spring then releases from the catch member as the stored charge launches the lancet to its lancing position. The leaf spring recharges towards the end of the lancing stroke, which return charge then retracts the lancet back into the housing.

### CROSS REFERENCE TO RELATED APPLICATIONS

This invention claims the priority benefit of U.S. Provisional Patent Application Ser. No. 60/574,779, filed on May 27, 2004, which is hereby incorporated herein by reference in its entirety for all purposes.

### TECHNICAL FIELD

The present invention relates generally to medical devices and, more particularly, to a lancing device for penetrating the skin of a human or animal subject for sampling of blood and/or other body fluids.

### BACKGROUND OF THE INVENTION

Lancing devices are used to penetrate the skin of a subject and obtain a sample of blood or other body fluid, as in the testing of blood sugar levels by diabetics. Typically, a lancet having a sharp point is translationally mounted within a housing portion of a lancing device. The lancet is driven by a spring or other biasing means to cause the sharp point to extend a small distance through an opening in the housing and into the subject's skin, creating a wound from which the sample of body fluid is collected. The housing optionally includes a pressure surface for “pumping” the wound to enhance sample size, and may also incorporate a capillary tube or other sample collection media. The endcap of the housing or a portion of the housing adjacent the lancet opening may include an open window or a transparent section for viewing the sample collection site, and may also include one or more sample size indicators for comparing the size of a sample to a desired sample size. Example lancing devices are shown in U.S. Pat. No. 5,356,420; U.S. Pat. No. 5,397,334; and U.S. Pat. No. 5,439,473, all of which are hereby incorporated herein by reference.

Lancing devices typically are intended either for a single use or for multiple uses. Single-use lancing devices generally are disposed of after one use. For example, in a hospital or clinic, it is desirable to provide a single-use lancing device that can be used on a patient and then disposed of to eliminate any risk of infection to subsequent patients or caregivers from exposure to residual body fluids remaining on the lancing device. Accordingly, single-use lancing devices oftentimes include a disabling mechanism to prevent accidental or intentional re-use of the device. Various forms of disabling mechanisms are available, and are well known in the art. For example, the disabling mechanism may comprise a return spring for retracting the sharp point of the lancet back into the housing after a single use, break-away elements or a frangible link in the cocking or triggering mechanism to prevent re-arming or re-firing the device after a single use, a locking element, and/or a shield for blocking travel of the lancet.

Because single-use lancing devices normally are disposed of after just one use, they generally are relatively simple in construction so that they can be economically manufactured in large quantities. But mass-producing a lancing device that is low in cost and at the same time safe, reliable, and not painful to use has proven difficult.

Accordingly, a need exists for low-cost lancing devices that are safe, reliable, and not painful to use. It is to the provision of lancing devices meeting these and other needs that the present invention is primarily directed.

### SUMMARY OF THE INVENTION

Briefly described, the present invention provides a lancing device having a lancing assembly and a housing assembly. The lancing assembly includes a cantilevered leaf spring arm that carries and drives a lancet through a lancing stroke, and an actuator element that drives the leaf spring arm. The housing assembly includes a catch surface that engages and restricts movement of the leaf spring arm for charging it, a lancing hole through which a lancing tip of the lancet extends to puncture the skin, and an enclosure for the lancet.

When the actuator element is in its set position, the leaf spring arm and lancet are in their set position with the leaf spring arm engaging the catch surface. When the actuator element is moved to its actuated position, the leaf spring arm pivots and deflects against the catch surface, thereby storing a drive charge. The leaf spring arm then releases from the catch surface as the drive charge launches the lancet to its lancing position. The leaf spring arm recharges towards the end of the lancing stroke, which return charge then retracts the lancet back into the enclosure and to its rest position.

In a first example embodiment of the present invention, the housing assembly includes a peripheral frame and two side panels that fold over open sides of the frame to form the enclosure for the lancet. A catch member extends inwardly from the frame to define the catch surface, and the frame and side panels are manufactured as a single integral piece. In addition, the actuator element is provided by an arm extending from the leaf spring arm, and the actuator arm, leaf spring arm, and lancet of the lancing assembly are manufactured as a single integral piece. In this way, the lancing device is constructed of only two pieces: the housing assembly and the lancing assembly. The lancing assembly snaps onto the housing assembly frame so that the leaf spring arm pivots within the enclosure.

The leaf spring arm is resilient and has a bend between a first segment and a second segment, with the lancet carried by the second segment. The bend allows the use of a longer leaf spring arm, which can be deflected more to store a greater charge. In addition, the second segment of the leaf spring arm is bowed upwards in its intermediate section, so that as this segment is deflected it is straightened out to increase its residence time on the catch member, thereby increasing the charge on the leaf spring arm. These aspects of the lancing assembly allow the housing assembly to be provided in a small size, while maintaining a fast lancet travel speed to minimize the pain felt by the lancet puncturing the skin.

In a second example embodiment, the housing assembly is made as a single piece with both side panels attached to the frame covering both sides of the frame, and with the top of the frame open. The lancing assembly is made as a single piece that drops into the enclosure through its open top. In addition, the catch surface is defined by a ledge formed in the frame 116.

In a third example embodiment, the housing assembly and the lancing assembly (thus, the entire lancing device) are manufactured integrally as a single piece, with the pivotal coupling of the lancing assembly to the housing assembly provided by a thinned wall in the single piece. In addition, the lancing device has a stop member that contacts a limit surface extending from the actuator element to limit the travel of the actuator arm back up to the first position after use, thereby functioning as a disabling mechanism to prevent reuse of the lancing device.

In a fourth example embodiment, the actuator arm and the leaf spring arm of the lancing assembly are provided by a single arm member that is preferably generally straight. And the frame includes a fulcrum member that engages and blocks the leaf spring arm from downward movement after the lancet punctures the skin in its lancing position, thereby functioning as a disabling mechanism to prevent reuse of the lancing device.

Accordingly, the lancing device may be provided as a single piece of plastic or other material, or as two pieces that are easily assembled together. In this way, the lancing device is very inexpensive to manufacture as a single-use disposable unit. It will be understood that the various advantageous aspects described herein may be combined in these or other ways to produce other low cost lancing devices that are within the scope of the present invention.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a side view of a lancing device according to a first example embodiment of the present invention, showing a housing assembly and lancing assembly in a set position.

FIG. 2 is a side view of the lancing device of FIG. 1, showing the lancing assembly in the midst of a lancing stroke.

FIG. 3 is a plan view of the housing assembly of FIG. 1, showing integral side panels being folded into place to form an enclosure.

FIG. 4 is a side view of a lancing device according to a second example embodiment of the present invention, showing a housing assembly (with one side panel removed) and a lancing assembly in a set position.

FIG. 5 is a side view of the lancing device of FIG. 4, showing the lancing assembly in the midst of a lancing stroke.

FIG. 6 is a plan view of the housing assembly of FIG. 4, showing integral side panels and an open top.

FIG. 7 is a left perspective view of a lancing device according to a third example embodiment of the present invention, showing a housing assembly and a lancing assembly in a set position.

FIG. 8 is a right perspective view of the lancing device of FIG. 7.

FIG. 9 is a side view of the lancing device of FIG. 7.

FIG. 10 is a side view of a lancing device according to a fourth example embodiment of the present invention, disproportionately large and with one side panel removed to show a lancing assembly traveling from a set position to a lancing position.

### DETAILED DESCRIPTION OF EXAMPLE EMBODIEMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

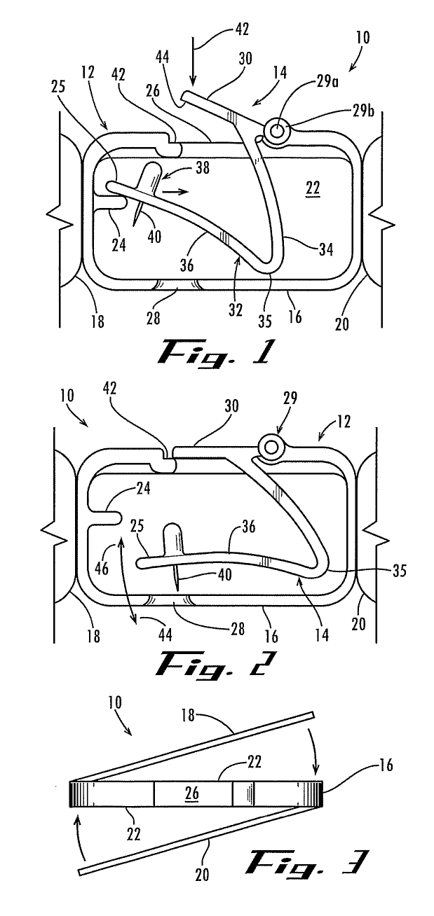

With reference now to the drawing figures, FIGS. 1-3 show a lancing device 10 according to a first example embodiment of the present invention. The lancing device 10 is a single use, disposable device. In alternative embodiments, the lancing device is adapted to accept replaceable lancets or lancing assemblies for use as a reusable lancing device.

The lancing device 10 includes a housing assembly 12 and a lancing assembly 14. The lancing device 10 is preferably constructed of molded plastic, though other materials and fabrication techniques may be suitably used.

The housing assembly 12 includes a peripheral frame 16 and two side panels 18 and 20 that cover the open sides of the frame to form an enclosure defining an interior space for a lancing tip of the lancing assembly 14. The frame 16 defines a catch surface 24, an actuator opening 26, and a lancing hole 28. The catch surface 24 is preferably defined by an arm extending inwardly from the frame 16, but alternatively it may be formed by another-shaped structure protruding from the frame, a notch recessed into the frame, or an offset in the frame that forms a ledge. The actuator opening 26 is sized and positioned for receiving therethrough a leaf spring arm of the lancing assembly. And the lancing hole 28 is sized and positioned for receiving therethrough the lancing tip for puncturing the skin.

In this embodiment, the lancing device 10 is constructed of only two pieces: the housing assembly 12 and the lancing assembly 14. The housing assembly frame 16 and side panels 18 and 20 are manufactured as a single integral piece. The frame 16 has two open sides 22 and the side panels 18 and 20 extend laterally from opposite ends of the frame 16 in a position exposing the open sides (see FIG. 1). And the side panels 18 and 20 are offset from each other, that is, they extend from the frame ends at opposite sides in a cater-corner arrangement (see FIG. 3). In this arrangement, the side panels 18 and 20 can be folded over and snapped in place (e.g., by a detent or keyed tab) to cover the open sides 22 (see FIG. 3). In alternative embodiments, the frame has one side closed by an integrally formed side panel and the other side open and closable by a foldable side panel extending from the frame, one or both of the side panels extend from a top or bottom or other part of the frame, or the frame and one or both of the side panels are manufactured as separate pieces and assembled together.

The lancing assembly 14 is pivotally coupled 29 to the frame 16. For example, the housing assembly 12 may include a pin 29a that is received in an eye 29b on the lancing assembly 14 (or vice versa, as in the second example embodiment described below) with a snap-fit. In alternative embodiments, the lancing assembly is pivotally coupled to the housing assembly by a hinge, a spring, a thinned/weakened wall section of the frame, and/or by another structure permitting pivotal movement.

The lancing assembly 14 includes an actuator element 30 and a cantilevered leaf spring arm 32. In this embodiment, the actuator element 30 and the cantilevered leaf spring arm 32 are manufactured as a single integral piece. In alternative embodiments, the actuator element 30 is a separate component that pivots or that moves in another fashion, such as a linear push-button that is operable to cause the leaf spring arm 32 deflect and pivot.

The actuator element 30 is preferably provided by an arm or other elongated member extending from the pivotal coupling 29. Operating the actuator arm 30 operates the leaf spring arm 32 to perform the lancing function.

The leaf spring arm 32 is resilient and has a bend 35 between a first segment 34 and a second segment 36. Preferably, the first and second segments 34 and 36 define an acute angle, which allows for the use of a longer leaf spring arm 32 (because it's bent back over on itself) housed within the enclosure than if the entire leaf spring arm was straight. Because the leaf spring arm 32 is longer, it can be deflected more to store a greater charge.

The first segment 34 extends from the actuator arm 30 (either directly or indirectly, e.g., with the pivotal coupling 29 therebetween), and the second segment 36 includes a lancet 38. The lancet 38 includes a lancing tip 40 (e.g., a needle or blade) for piercing the skin. In a set position, the second segment 36 engages the catch surface 24 with the lancing tip 40 inside the enclosure defined by the housing assembly 12. Preferably, the catch surface 24 is engaged by an end 25 of the second segment 36, with the lancing tip 40 between the end of the leaf spring arm 32 and the pivotal coupling 29, but closer to the end.

When the lancing device 10 is actuated, the leaf spring arm 32, or at least the second segment 36 of it, resiliently deflects and charges until it releases from the catch surface 24 to travel to a lancing position with the lancing tip 40 extending through the lancing hole 28, as described next. The second segment 36 may be curved to increase the charging of the leaf spring arm 32. For example, the second segment 36 may be bowed up slightly at about its intermediate section, as shown. Thus, when in the rest position of FIG. 2, the distal end 25 and the bend 35 are lower than the intermediate section of the second segment 36 between them. In this configuration, as the leaf spring arm 32 deflects it is straightened out and thereby effectively lengthened slightly. So as the leaf spring arm 32 pivots the end 25 of the second segment 36 away from engagement with the catch surface 24, the end and catch surface remain in contact slightly longer, thereby allowing the leaf spring arm 32 to receive a greater charge. Alternatively, the end 25 of the second segment 36 may be turned downwardly to achieve the same function.

In operation, before use the actuator arm 30 is in its set position and the leaf spring arm 32 in its set position with the end 25 of the second segment 36 engaging the catch surface 24, as shown in FIG. 1. To activate the lancing device 10, the actuator arm 30 is depressed, for example, by a user's thumb (see directional arrow 42 in FIG. 1) and pivoted from its set position towards its actuated position. This force urges the leaf spring arm 32 to pivot, but because the second segment 36 is engaged by the catch surface 24, the leaf spring arm 32 (particularly the second segment 36) deflects and thereby charges. As the actuator arm 30 is depressed farther towards its actuated position, the leaf spring arm 32 deflects farther and pivots away from the catch surface 24 until the second segment end 25 slides inwardly off and out of engagement with the catch surface. Then the drive charge stored by the leaf spring arm 32 launches the lancet 38 on the second segment 36 into its lancing stroke towards its lancing position (see directional arrowhead 44 in FIG. 2). In the lancing position, the lancing tip 40 extends through the lancing hole 28 and punctures the skin.

Before the lancing tip 40 reaches the lancing position, the leaf spring arm 32 discharges, but its momentum keeps it traveling until it reaches the lancing position. During this portion of its travel the leaf spring arm 32 is charging a second time. The lancing tip 40 momentarily stops at the lancing position due to a mechanical stop (the leaf spring arm 32 contacting the interior wall of the frame 16) and/or its momentum being spent. The lancing tip 40 is then retracted (see directional arrowhead 46 in FIG. 2) back into the enclosure as the leaf spring arm's second/return charge discharges. And the lancet 38 then oscillates slightly within the enclosure until it comes safely to its rest position shown in FIG. 2.

The frame 16 has a stop surface 42 that is contacted by a limit surface 44 of the actuator arm 30 to limit the travel of the actuator arm at the actuated position. With the actuator arm 30 stopped at its actuated position and the leaf spring arm 30 at its rest position, the lancing tip 40 is contained safely within the enclosure. The leaf spring arm 32 is configured (resiliency, bend angle, lancing tip length, etc.) so that the lancing tip 40 stays within the enclosure and does not extend through the lancing hole 28, so prevent accidental stickings and reuse of the device 10. In alternative embodiments, the lancing device 10 is provided with a lock mechanism or a disabling mechanism to secure the lancing tip 40 within the enclosure after one use.

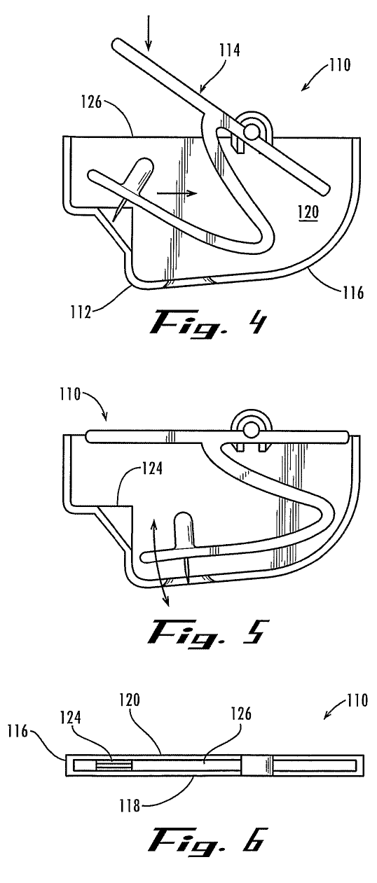

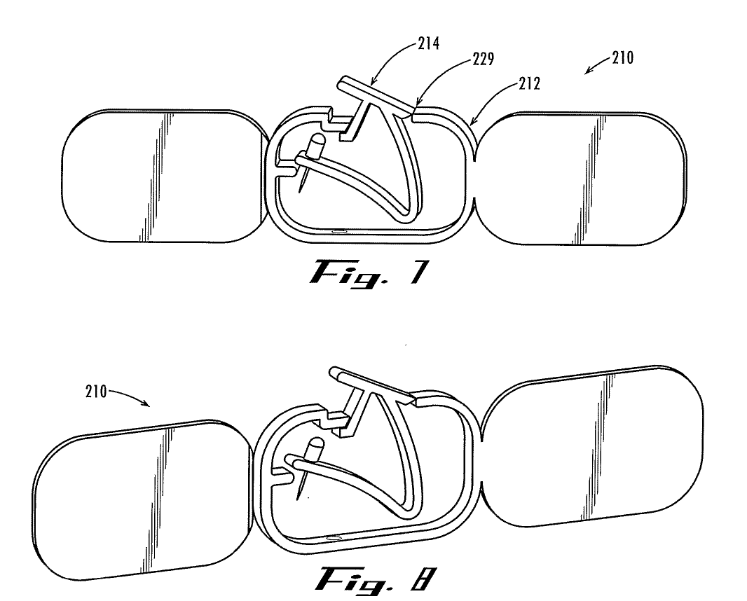

Referring to FIGS. 4-6, there is shown a lancing device 110 according to a second example embodiment of the present invention. The lancing device 110 is similar to that of the first embodiment and has a housing assembly 112 and a lancing assembly 114. But the housing assembly 112 has a frame 116 with an open top 126 and with both sides closed by side panels 118 and 120 that are integrally formed with the frame 116 to form the enclosure. And the actuator arm 130 preferably extends substantially the length of the open top 126 of the frame 116 to substantially cover it when in the rest position of FIG. 5. The longer actuator arm 130 preferably includes a cover end opposite of the where the actuator arm is depressed, with the pivotal coupling therebetween. In addition, the catch surface 124 is defined by an offset/ledge formed in the frame 116. For example, the catch surface 124 may be formed by two opposing ledges extending inwardly from the sides 118 and 120 with a gap between them for the lancing tip to pass through. Otherwise, the structure and operation of the lancing device 110 of the second embodiment are the same as or similar to that of the lancing device 10 of the first embodiment. In alternative embodiments, the frame includes a stop surface for engaging and stopping the actuator arm travel at the actuated position, to retain the lancing tip in the housing after one use.

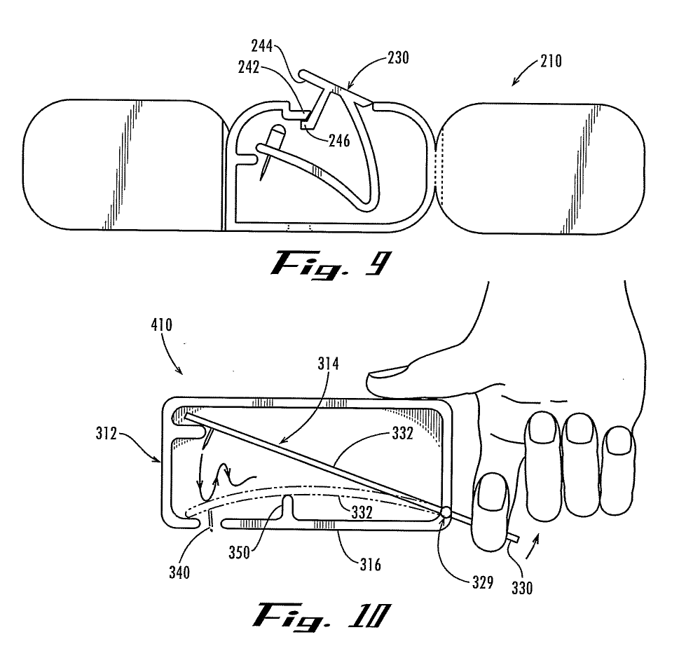

Referring to FIGS. 7-9, there is shown a lancing device 210 according to a third example embodiment of the present invention. The lancing device 210 is similar to that of the first embodiment and has a housing assembly 212 and a lancing assembly 214. But in this embodiment the housing assembly 212 and the lancing assembly 214 (thus, the entire lancing device 210) are integrally formed as a single molded piece. The pivot point 229 between the lancing assembly 214 and the housing assembly 212 is provided by a thinned wall in the single molded piece that permits pivoting by the lancing assembly relative to the housing assembly. The thinned wall may define a notch or other recess that is weaker and thus deflects to permit the pivoting motion.

In addition, the lancing device 210 has a stop member 242 that contacts a first limit surface 244 to limit the travel of the actuator arm 230 when actuated. The stop member 242 also contacts a second limit surface 246 extending from the actuator arm 230 to limit the travel of the actuator arm back up to the first position after use. Once the device 210 has been actuated for lancing, the second limit surface 246 prevents the actuator arm 230 from being pulled back up far enough to pull the second segment of the leaf spring arm back up past the catch member to reset the device. Thus, the stop member 242 and the second limit surface 246 cooperatively function as a disabling mechanism to prevent reuse of the device 210. Otherwise, the structure and operation of the lancing device 210 of the third embodiment are the same as or similar to that of the lancing device 10 of the first second embodiment.

Referring to FIG. 10, there is shown a lancing device 310 according to a fourth example embodiment of the present invention. The lancing device 310 is similar to those of the other embodiments described herein and has a housing assembly 312 and a lancing assembly 314. But the actuator arm 330 and the leaf spring arm 332 of the lancing assembly 314 are provided by a single generally straight member (without the bend) that is pivotally coupled 329 to the end of the frame 316 (not the top). Alternatively, the actuator arm 330 and the leaf spring arm 332 may be angled somewhat relative to each other for better ergonomics, while still having a simple design that is easy and inexpensive to manufacture. The actuator arm 330 is pulled up instead of pushed down to actuate the device 310. And the frame 316 includes a fulcrum member 350 extending inwardly therefrom and about which the leaf spring arm 332 deflects in the lancing position (shown in broken lines). When the leaf spring arm 332 is in the rest position after use, the fulcrum member 350 engages it and blocks it from downward movement, thereby functioning as a disabling mechanism to retain the lancing tip 340 in the enclosure to prevent reuse of the device 210.

While the invention has been described with reference to preferred and example embodiments, it will be understood by those skilled in the art that a variety of modifications, additions and deletions are within the scope of the invention, as defined by the following claims.

1. A lancing device, comprising:
a housing assembly forming an enclosure and defining a catch surface;
a leaf spring arm;
a lancet that is carried by the leaf spring arm and has a lancet tip; and an actuator element that is operable to move the leaf spring arm so that, from a set position engaging the catch surface, the leaf spring arm deflects against the catch surface to store a drive charge, moves out of engagement with the catch surface, and is propelled by the drive charge to a lancing position with the lancet tip extending out of the enclosure.

2. The lancing device of claim 1, wherein the leaf spring arm has a bend between a first segment and a second segment.

3. The lancing device of claim 2, wherein the first segment and the second segment define an acute angle.

4. The lancing device of claim 2, wherein the lancet is positioned on the second segment and the second segment has a curvature between the bend and the lancet.

5. The lancing device of claim 1, wherein the actuator element comprises a pivotal actuator arm from which the leaf spring arm extends, wherein the actuator arm and the leaf spring arm pivot together.

6. The lancing device of claim 5, wherein the actuator arm and the leaf spring arm are generally linear.

7. The lancing device of claim 5, wherein the actuator arm travels from a set position to an actuated position to operate the lancing device, the actuator arm includes a first limit surface, and the housing assembly includes a stop member that is engaged by the first limit surface to limit the travel of the actuator arm at the actuated position.

8. The lancing device of claim 7, further comprising a second limit surface that engages the stop member to limit the return travel of the actuator to the set position so that the leaf spring is disabled from being returned back past the catch surface to the set position.

9. The lancing device of claim 7, wherein the leaf spring arm is configured to, before reaching the lancing position, discharge and deflect to store a return charge that retracts the lancet from the lancing position to a rest position, wherein the lancet tip is within the enclosure when the actuator is in the actuated position and the lancet is in the rest position.

10. The lancing device of claim 1, wherein the leaf spring arm is configured to, before reaching the lancing position, discharge and deflect to store a return charge, wherein the leaf spring arm drives the lancet from the set position to the lancing position, and then the leaf spring arm retracts the lancet from the lancing position to a rest position within the enclosure.

11. The lancing device of claim 1, wherein the housing assembly includes an inwardly extending arm or a ledge that defines the catch surface.

12. The lancing device of claim 1, wherein the housing assembly includes two side panels each having a ledge extending inwardly therefrom and defining the catch surface, wherein the two ledges define a gap therebetween through which the lancet tip passes when traveling from the set position to the lancing position.

13. The lancing device of claim 1, wherein the housing assembly includes an inwardly extending fulcrum member about which the leaf spring deflects in the lancing position and that blocks the lancet tip from extending out of the enclosure when the lancet comes to stop in a rest position.

14. The lancing device of claim 1, wherein the housing assembly comprises a frame and two side panels that form the enclosure and are integrally formed as a single piece, and wherein the actuator arm, the leaf spring arm, and the lancet are integrally formed as a single piece that defines a lancing assembly, wherein the lancing device has a two-piece construction consisting only of the housing assembly and the lancing assembly.

15. The lancing device of claim 14, wherein the frame has at least one open side and at least one of the side panels extends from the frame in a position exposing the open side and is foldable to cover the open side to form the enclosure.

16. The lancing device of claim 14, wherein the frame has an open top that receives the leaf spring arm to assemble the lancing device.

17. The lancing device of claim 1, wherein the housing assembly comprises a frame and two side panels that form the enclosure, wherein at least one of the side panels is manufactured in a position exposing an open side of the frame and is foldable to cover the open side, wherein the frame, the side panels, the actuator arm, the leaf spring arm, and the lancet are all integrally formed as a single piece, and wherein the leaf spring arm pivots about a pivot point defined by a thinned wall section of the lancing device.

18. A lancing device, comprising:
a housing assembly forming an enclosure and defining a catch surface; and
a lancing assembly including an actuator arm, a leaf spring arm extending from the actuator arm, and a lancet that is carried by the leaf spring arm and has a lancet tip, the leaf spring arm having a first segment and a second segment with a bend therebetween and defining an acute angle, the leaf spring arm pivotally moveable relative to the enclosure,
wherein the actuator arm is pivotal to pivot the leaf spring arm so that, from a set position engaging the catch surface, the leaf spring arm deflects against the catch surface to store a drive charge, pivots out of engagement with the catch surface, and is propelled by the drive charge to a lancing position with the lancet tip extending out of the enclosure, and
wherein the leaf spring arm is configured to, before reaching the lancing position, discharge and deflect to store a return charge that retracts the lancet from the lancing position to a rest position with the lancet tip within the enclosure.

19. The lancing device of claim 18, wherein the actuator arm travels from a set position to an actuated position to operate the lancing device, the actuator arm includes a first limit surface, and the housing assembly includes a stop member that is engaged by the first limit surface to limit the travel of the actuator arm at the actuated position, wherein the lancet tip is within the enclosure when the actuator is in the actuated position and the lancet is in the rest position.

20. The lancing device of claim 18, wherein the housing assembly comprises a frame and two side panels that form the enclosure and are integrally formed as a single piece, and the actuator arm, leaf spring arm, and lancet are integrally formed as a single piece that defines a lancing assembly, wherein the lancing device is of a two-piece construction, the two pieces being the housing assembly and the lancing assembly.

21. The lancing device of claim 18, wherein the housing assembly comprises a frame and two side panels that form the enclosure, wherein at least one of the side panels is manufactured in a position exposing an open side of the frame and is foldable to cover the open side, wherein the frame, the side panels, the actuator arm, the leaf spring arm, and the lancet are all integrally formed as a single piece, and wherein the leaf spring pivots about a pivot point defined by a thinned wall section of the lancing device.

22. A lancing device, comprising:
an integrally formed, one-piece housing assembly forming an enclosure that defines a catch surface; and
an integrally formed, one-piece lancing assembly comprising an actuator arm, a leaf spring arm extending from the actuator arm, and a lancet that is carried by the leaf spring arm and has a lancet tip, the lancing assembly mounted to the housing assembly to permit pivotal movement of the leaf spring arm,
wherein the actuator arm is operable to pivot the leaf spring arm so that, from a set position engaging the catch surface, the leaf spring arm deflects against the catch surface to store a drive charge, pivots out of engagement with the catch surface, and is propelled by the drive charge to a lancing position with the lancet tip extending out of the enclosure, and
wherein the lancing device is of a two-piece construction, the two pieces being the housing assembly and the lancing assembly.

23. The lancing device of claim 22, wherein the housing assembly comprises a frame having at least one open side through which the leaf spring arm is inserted when mounting the lancing assembly to the housing assembly during assembly of the two-piece lancing device, and at least one side panel extending from the frame in a position exposing the open side and foldable to cover the open side to form the enclosure.

24. The lancing device of claim 22, wherein the housing assembly comprises a frame having two open sides and two side panels extending from the frame in a position exposing the open sides and foldable to cover the open sides to form the enclosure.

25. The lancing device of claim 24, wherein the foldable side panels extend from the frame in a cater-corner arrangement.

26. The lancing device of claim 22, wherein the housing assembly comprises a frame having an open top through which the leaf spring arm is inserted when mounting the lancing assembly to the housing assembly during assembly of the two-piece lancing device.

27. The lancing device of claim 26, wherein the actuator arm has a length to substantially cover the open top of the enclosure.

28. A lancing device, comprising:
an integrally formed, one-piece housing assembly and lancing assembly;
the housing assembly forming an enclosure that defines a catch surface;
the lancing assembly comprising an actuator arm, a leaf spring arm extending from the actuator arm, and a lancet that is carried by the leaf spring arm and has a lancet tip; and
the lancing assembly pivotally moveable relative to the housing assembly at a pivot point between the lancing assembly and the housing assembly,
wherein the actuator arm is operable to pivot the leaf spring arm so that, from a set position engaging the catch surface, the leaf spring arm deflects against the catch surface to store a drive charge, pivots out of engagement with the catch surface, and is propelled by the drive charge to a lancing position with the lancet tip extending out of the enclosure.

29. The lancing device of claim 28, wherein the housing assembly comprises a frame having two open sides and two side panels extending therefrom in a position exposing the open sides and foldable to cover the open sides to form the enclosure.

30. The lancing device of claim 29, wherein the foldable side panels extend from the frame in a cater-corner arrangement.

31. The lancing device of claim 28, wherein the pivot point comprises a thinned wall section of the lancing device.
